(19) United States
(12) Patent Application Publication
(10) Pub. No.: US 2017/0231555 A1
(43) Pub. Date: Aug. 17, 2017

(54) DEVICE FOR MONITORING PHYSIOLOGICAL PARAMETERS

(71) Applicant: The Research Foundation for The State University of New York, Albany, NY (US)

(72) Inventors: Craig Lehmann, Aquebogue, NY (US); John Brittelli, Brookhaven, NY (US); Lisa Johnson, Amityville, NY (US); Sharon Martino, Stony Brook, NY (US); Michael Gouzman, Centereach, NY (US)

(73) Assignee: The Research Foundation for The State University of New York, Albany, NY (US)

(21) Appl. No.: 15/583,329

(22) Filed: May 1, 2017

Related U.S. Application Data
(63) Continuation of application No. 14/427,060, filed on Mar. 10, 2015.
(60) Provisional application No. 61/699,663, filed on Sep. 11, 2012.

(51) Int. Cl. A61B 5/00; A61B 5/08

(52) U.S. Cl. CPC A61B 5/4818; A61B 5/0816; A61B 5/486; A61B 5/746; A61B 5/743; A61B 5/6823; A61B 5/6831; A61B 5/6833; A61B 5/0024; A61B 5/6826; A61B 5/6829; A61B 5/7275; A61B 5/7405; A61B 5/7278; A61B 5/7282; A61B 2560/0214; A61B 2562/0219; A61B 2503/04; A61B 2503/12

(57) ABSTRACT

A physiology monitoring system is provided for reducing the risk of sudden unexpected infant death in infants and to monitor physiological parameters in both infants and adults. An embodiment of the monitoring system includes a belt dimensioned to encircle a patient's chest; an accelerometer configured to detect inhale/exhale motions of the patient's chest; a gyroscopic sensor configured to detect supine/prone orientation of the patient; a controller configured for receiving signals from the accelerometer and gyroscopic sensor and determining a risk to the patient based on the received signals; and an e-paper display. The e-paper display being coupled to the control circuit and configured to display instructional messages to a caregiver. Additionally, a power source provides energizing power to the accelerometer, gyroscopic sensor, controller and e-paper display for a minimum period of one year.

### CROSS-REFERENCE TO RELATED REFERENCES

The present application is a continuation of a co--pending application having U.S. Ser. No. 14/427,060, filed on Mar. 10, 2015, which is a 371 of International application having Serial No. PCT/US20131059141, filed on Sep. 11, 2013, which claims the benefit of priority from U.S. Provisional Patent Application No. 61/699,663 filed on Sep. 11, 2012, the entire contents of all of which are incorporated herein by reference.

### FIELD OF THE INVENTION

The present invention relates generally to infant monitoring devices. More specifically, the present invention relates to a system and method for reducing sudden unexpected infant death.

### BACKGROUND OF THE DISCLOSURE

Sudden Unexpected Infant Death (SUID) is a leading cause of death worldwide for children less than one year of age. The incidence of SUM is 25 times higher in underdeveloped countries. Of the ten countries worldwide with the highest SUID rates, nine are in Africa (US Central Intelligence Agency). Infants in Kenya die at a rate of 121 deaths per 1000 live births (United Nations). More than 1 in 10 infants in Kenya die of SUID. Research indicates a variety of controllable factors that influence the incidence of SUID: (1) monitoring for apnea; (2) sleeping position (an infant should sleep supine, not prone); and (3) environment (e.g. mattress should be firm).

Apnea monitors currently in use are ill suited for the developing world because they are expensive, complex to operate, consist of many parts that can break, and require an external power source. These devices are not feasible in resource-poor areas where electricity is unstable or absent, and where education, training and language can be barriers to proper use. The monitors do not address body position nor do they provide caregiver instruction.

Currently, no solutions exist that address the need for low cost, easy to use, well-designed monitors that do not require an external power source.

### SUMMARY OF THE DISCLOSURE

An object of the present invention is to provide a simple, low-cost infant monitor that will reduce the controllable factors associated with SUID by monitoring infants' breathing and sleeping position and by raising caregiver awareness of SUID risk factors. This one-piece monitor will have an intuitive design so that it can be operated by anyone, anywhere, regardless of education, language or training.

Present invention has the potential to save infants who are vulnerable to SUID due to risk factors such as low birth weight and history of respiratory distress, as well as cultural and educational issues surround sleeping position and environment. The monitor of the present invention s a unique combination of low-cost, state of the art technology and universal design principles. The project has worldwide applicability and scalability and will result in a monitor that will be suited for hospital, healthcare facility or home use.

An embodiment of the present invention includes: a belt dimensioned to encircle an infant's chest; an accelerometer configured to detect inhale/exhale motions of the infant's chest, the accelerometer being disposed on the belt; a gyroscopic sensor configured to detect supine/prone orientation of the infant, the gyroscopic sensor being disposed on the belt; a controller configured for receiving signals from the accelerometer and gyroscopic sensor and determining a risk to the infant based on the received signals; an e-paper display disposed at a portion of the belt, the e-paper display being coupled to the control circuit and configured to display instructional messages to a caregiver; and a power source disposed within the belt, the self-contained power source providing energizing power to the accelerometer, gyroscopic sensor, control circuit and e-paper display for a minimum period of one year.

### BRIEF DESCRIPTION OF THE DRAWINGS

These and other features, aspects, and advantages of the present invention will become better understood with regard to the following description, appended claims, and accompanying drawings wherein:

FIG. 1 illustrates a block representation of an embodiment of the present invention;

FIG. 2 illustrates a flow diagram of a monitoring process performed by an embodiment of the present invention; and

FIG. 3 illustrates a block representation of an embodiment of the present invention.

### DETAILED DESCRIPTION OF DISCLOSURE

The present invention decreases the incidence of SUM by monitoring breathing, monitoring body position and providing advice to a caregiver. The monitor 100 of the present invention is within a single fabric belt 102 that is secured around the infant's chest before he or she goes to sleep. The fabric belt 102 contains a gyroscopic sensor 104 to detect the baby's body position, and an accelerometer 106 to detect changes in chest wall movement (a reliable indicator of breathing). Additionally, a flexible “e-paper” screen 108 is disposed on a surface of the fabric belt 102 to display pictogram instructions. A long-life battery 114 having an estimated 12 month lifespan, which exceeds the period of time considered critical for SUID, is provided in the fabric belt 102 as well.

The battery may be a Lithium polymer battery, or any other type of battery capable of maintaining adequate charge for powering the electronic components and sensors for a minimum of one year. Additionally, any appropriate power source, for example a fuel cell or other energy storage device as known in the art, may be used in place of batteries.

When the monitor 100 detects a risk factor (drop in respiration; baby rolls over), audible and visual alarms emitted by speakers 110 and light emitting diodes (LED) 112, respectively, will alert the caregiver immediately. Additionally, the screen 108 will provide clear instructions on actions to be taken to correct the risk factor. The screen provides simple instructions, preferably in the form of pictographs, for avoiding risk factors. For example, a “sad face” may be displayed if the child is positioned to sleep on his or her stomach. By providing instructions in the form of pictographs, the present invention is intended to be language and education-level independent.

A control system 116 receives signals from the gyroscopic sensor 104 and accelerometer 106 and monitors the physiological conditions of the infant, including respiratory rate and space orientation (vertical or horizontal and lying on back or face down) The control system 116 may employ a low-power microcontroller, application specific integrated circuit (ASIC), system-on-chip (SoC), system in package (SiP), or field programmable gate array (FPGA).

In one embodiment, the internal electronic system will include a set of thin-film based sensors, an e-paper flexible display with touch sensitive function, a flat and flexible Li-polymer battery, ultra-low power microcontroller and a set of notification components, such as lights, sound, and optional Bluetooth 4 wireless interface. No buttons switches or external contacts are necessary in the present invention. However, a magnetic strip, acting as an initiator, 118a is removably disposed on an external surface of the fabric belt 102. When the initiator 118a is removed, internal magnetic switch 118b closes, which will energize the monitor 100. Once energized, the monitor 100 of the present invention cannot be switched off.

In addition the electronics and sensors are hermetically sealed within the fabric belt 102. Thus, the monitor 100 will be water proof, dust proof, and will contain no external parts, wires, knobs, nor dials.

An embodiment of the present invention is implemented as a lightweight multi-layer structure securely encased within the belt. The electronic components can he integrated in a single ASIC. A laser-etched Printed Circuit Board will be implemented, for example, on 25 micron Kapton film. Total weight of the monitor 100 is preferably between 50 g to 100 g.

The black and white e-paper type display can include a touch sensitive functionality in an embodiment of the present invention. However, in its simplest form, the monitor 100 of the present invention does not require touch sensitive functionality.

Feedback sensors will prompt event guidance for the caregiver. In order to avoid usage of the monitor 100 once the battery 114 has been drained, end-of-lifetime for the monitor is permanently indicated on the display with a simple pictogram. The monitor 100 is beneficial for all newborns, but critical for medically vulnerable infants during their first year of life.

In an embodiment of the present invention, an interactive educational system for teaching caregivers techniques for reducing risk factors may be provided. The interactive educational system may be menu driven and accessible by way of the e-paper screen 108 equipped with touch sensitive functionality.

Additionally, the interactive educational system provides an interactive educational and information. function based on a cause of an alert, a particular algorithm can activate a visual or audio channel of communication with a caregiver to provide guidance for correcting the risk factor or even provide first aid treatment to the infant if necessary.

Additionally, embodiments of the present invention have one or more of Bluetooth, 802.11a/b/g/n (WiFi) and cellular connectivity provided through a communication circuit 120 disposed within the fabric belt 102. In such an embodiment, Bluetooth enabled devices and WiFi enabled devices can be configured to receive data from the monitor 100. Additionally, the Bluetooth devices and WiFi devices can be used to browse, or access the interactive educational system stored in non-volatile memory 122 disposed on the fabric belt 102. Such non-volatile memory 122 may be formed from flash memory (either NAND-type or NOR-type), for example.

In addition, embodiments of the present invention which are equipped with a cellular-enabled communication circuit 120 can be configured to automatically contact first responders, such as a local emergency medical service, fire department, police department, or emergent care facility, upon detection of a serious risk factor. Serious risk factors may be conditions which a caregiver cannot be reasonably expect to properly handle, such as abnormal heart rate/rhythm, lack of breathing, and skin temperature elevated beyond a preset threshold, such as 103° F. for example. Other serious risk factors may be included, as well, depending on the availability of a monitoring component for inclusion in the monitor 100. The monitor 100 is configured in this embodiment to transmit relevant data to the first responders and allow the first responders to provide vocal instructions to a caregiver. The monitor 100 may be provided with a GPS system disposed within the fabric belt 102 as well, which, when coupled with the present embodiment, can provide first responders with a location of the infant in distress without relying on the caregiver.

Additionally, the monitor 100 may provide, by way of a touch sensitive e-paper screen 108 and cellular-enabled communication circuit 120, the ability for a caregiver to contact a helpline or pediatric physician manually with concerns or health related questions. In this embodiment, a microphone (not shown) is provided to facilitate two-way voice communication.

In addition to storing the interactive educational system, the non-volatile memory 122 can be configured to store configuration data used by the controller 116 to determine whether a risk factor is encountered based on the received sensor signals. The configuration data may be threshold values, for example, for acceptable respiration rate as measured by the accelerometer 106, and for acceptable range of angles as measured by the gyroscopic sensor 104. Alternatively, the thresholds may be implemented in hardware via the use of logic gates and the like, as well known in the art.

In an embodiment of the present invention, the control system 116 is a computer controlled adaptive network of sensors and data conditions. In an embodiment, two networks of sensors (health sensor network and environmental sensor network) are provided, which have constant, but different temporal monitoring rates.

The health sensor network for monitoring infant health is a fast data logging system. The health sensor network includes one or more sensors, such as breath detection means, for example an accelerometer, orientation detection means, for example a three-dimensional gyroscope, skin temperature sensors, heart rate sensor, heart rhythm monitor, carboxy-hemoglobin sensor, blood hemoglobin level sensor, perfusion index, and pulse oximetry sensor.

The environmental sensor network, which monitors environmental conditions, is a slow data logging system. The environmental sensor network can monitor a multitude of known controllable risk factors such as ambient temperature, carbon monoxide level, humidity, alcohol, and direct sunlight.

The above embodiments of the present invention are described as incorporated into a fabric belt 102, however one of ordinary skill in the art can readily envision modifications of the present invention in which the various components and sensors described above are incorporated into an infant undergarment, swaddle, or other wearable device. When the present invention is incorporated into an undergarment such as an infant bodysuit, commonly referred to as an onesie, additional sensors can be added and positioned at more appropriate locations on the infant's body. For example, a skin temperature sensor may be provided at a location near the infant's liver.

Also, auxiliary sensors may be deployed on separate straps, belts or rings that are fastenable to an infant's finger or toe. The auxiliary sensors further include Bluetooth, or other appropriate communication circuits, for wirelessly communicating data to the monitor 100. For example, sensors for measuring carboxyhemoglobin, blood hemoglobin level, perfusion index, and mike oximetry, and heart rate are best placed on an infant's toe. Thus, by providing these auxiliary sensors remotely located from the fabric belt 102, the present invention allows for more complete monitoring of the infant,

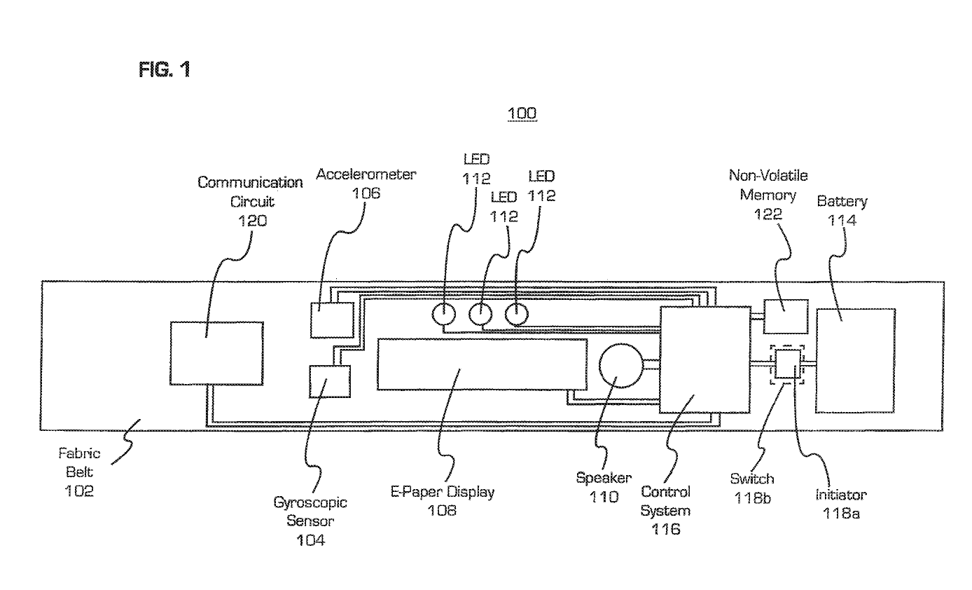

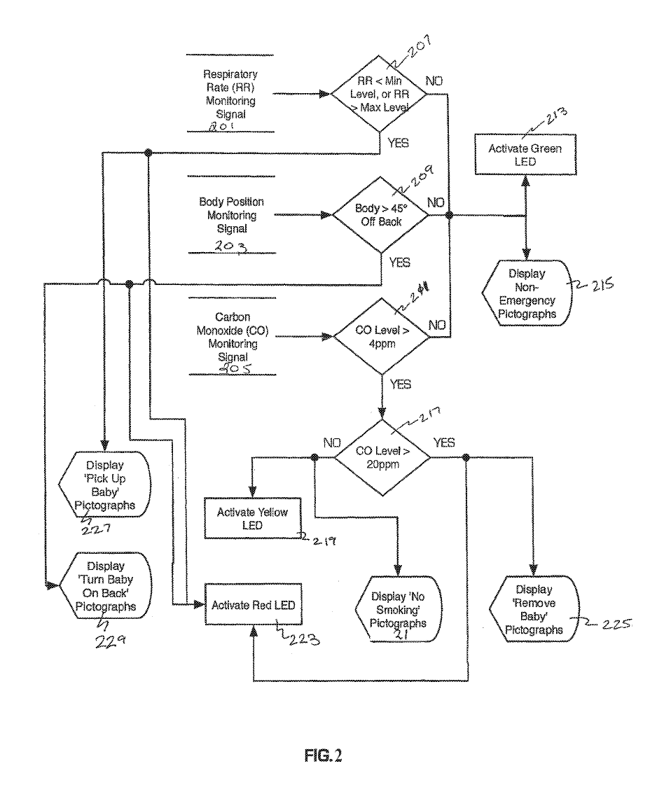

Turning to FIG. 2, a flow diagram is shown illustrating a monitoring and alert process performed by an embodiment of the present invention. Initially, a control system, such as the control system 116 shown in FIG. 1, receives signals from a respiratory rate sensor 201, body position sensor 203, and a carbon monoxide sensor 205. Each signal is individually compared with an appropriate threshold value. Thus, if the respiratory rate is above a minimum level and below a maximum level in step 207, the control system activates a green LED denoting that the infant s not at risk in step 213. Similarly, if the body position signals indicate that the infant is no more than 45° off his or her back in step 209, the control system activates a green LED denoting that the infant is not at risk in step 213. If the carbon monoxide signals indicate levels below 4 ppm in step 211, the control system activates a green LED denoting that the infant is not at risk in step 213. Additionally, while in this no risk state, the e-paper display may show one or more pictograms indicative of proper ways to reduce SUID risks, or other non-emergency related pictographs.

However. If any of the signals are outside the acceptable ranges in steps 207, 209 and 211, the process does not activate the green LED in step 213 or proceed to step 215. instead, the process proceeds as follows.

In the case where the respiratory rate is either below the minimum level or above the maximum level in step 207, the Red LED is activated in step 223 and the e-paper display shows a pictograph indicating that the caregiver should pick up the infant in step 227. Additionally, an alarm may sound in combination with the red LED being activated in step 223.

Similarly, if the infant is detected to have rotated more than 45° off his or her back in step 209, the, the Red LED is activated in step 223 and the e-paper display shows a pictograph indicating that the caregiver should turn the infant onto his back in step 229.

If the carbon monoxide level is detected above 4 ppm in step 211, and below 20 ppm in step 217, the process activates a yellow LED in step 219. Additionally, the e-paper display shows a pictograph indicating no smoking in step 221. However, if step 217 indicates that the carbon monoxide level is above 20 ppm, the Red LED is activated in step 223 and the e-paper display shows a pictograph indicating that the caregiver should remove the infant from the area in step 225.

The table below provides an example set of parameters that may be monitored by embodiments of the present invention, either individually or in combination. Additionally, the table provides an example monitoring instrument and the thresholds that trigger an alert.

TABLE 1

| Parameter | Instrument | Warning Threshold | Alarm Threshold |
|---|---|---|---|
| Heart Rate | Voltage Probe | <100 beats/min | <60 or >200 bpm |
| Respiration | Accelerometer | No breaths | No breaths |
| | | detected for | detected for |
| | | 10 sec, or less | 20 sec, or less |
| | | than 14 breaths | than 10 breaths |
| | | over 1 min | over 1 min |
| Body Orientation | 3-D Gyroscope/ | 45° rotation | 90-180° rotation |
| | Accelerometer | | |
| Perfusion Index | Optical Sensor | Monitor only | |
| Blood | Optical Sensor | Alarm only | <9 or >16 mg/dl |
| Hemoglobin | | | |
| Carboxy- | Optical Sensor | 3% COHB | 4% COHb |
| Hemoglobin | | | |
| Pulse Oximetry | Optical Sensor | <90 | <85% |
| Skin Temperature | Thermistor | N/A | >100° F. |
| Ambient | Thermistor | <95 | >100° F. |
| Temperature | | | |
| Ambient CO | CO sensor | >4 ppm | >15 ppm |

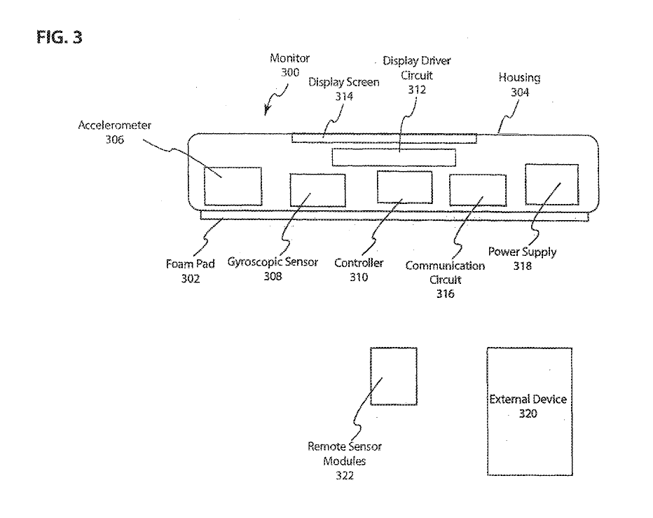

In an embodiment of the present invention as shown in FIG. 3, the monitor 300 is configured as a stick-on device or fob. Herein, a fob is understood to refer to a small self-contained device dimensioned and shaped similar to automotive alarm fobs for example. However, in the context of the present invention a fob is understood to be adhereable to a patient's skin rather than worn on a keychain or the like as in the case of an automobile alarm fob.

The monitor 300 includes a foam pad 302 disposed on an underside of the monitor 300, which is coated in an adhesive appropriate for removably contacting human skin, such as hydrogel adhesives used in commonly found disposable electrocardiogram electrodes. The monitor 300 contains within its housing 304 at least an accelerometer 306, gyroscopic sensor 308, controller 310. In addition the monitor 300 may include a display driver circuit 312 controlled by the controller 310 and coupled to a display screen 314. The display screen 314 may be electrophoretic (e-paper), OLED (organic LED), or LCD, and have a touch sensitive surface. The display screen 314 is disposed on a surface of the monitor housing 304 or within the housing 304 in line with an aperture formed on the surface of the housing 304 through which at least a majority of the display screen 314 is visible.

Moreover, the monitor 300 includes a communications circuit 316 which provides wireless connectivity by way of Bluetooth, or other short or medium range wireless communication protocols, and a power supply 318, such as a battery for example, configured to power the various circuits and components of the monitor 300 for a period of at least a year of continuous use in one embodiment. The communication circuit 316 is configured to communicate with various external devices 320, for example mobile phones, personal digital assistants (PDA), tablets, laptop and desktop computers. Also, the communication circuit 316 may be configured to communicate with additional remote sensor modules 322.

While the above embodiments have been described with respect to use on an infant, the present invention can be applied equally to adult patients in hospitals, at home, and in hospices. When applied to adult patients, the embodiment shown in FIG. 1 has a fabric belt 102 that is dimensioned to encircle an adult about the thorax region. With respect to FIG. 3, no appreciable alteration to the physical structure of the monitor 300 is necessary to configure the embodiment for use on an adult.

When using the present invention on an adult, a different sub-set of the parameters listed above in TABLE 1 may be monitored as appropriate. Additionally, the trigger points for issuing alarms may be altered to correspond to adult physiology. Moreover, the interactive educational system can be tailored to adult patients, and may be farther tailored to an individual patient based on the patient's medical condition and history.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. A physiology monitor, comprising:
a housing comprising:
an accelerometer configured to:
monitor a respiratory rate of a patient and generate respiratory rate data based on the monitored respiratory rate of the patient; and
monitor an orientation of the patient and generate orientation data based on the monitored orientation of the patient;
a display device disposed at a portion of the housing, the display device configured to display pictographic instructions to a caregiver;
a controller in communication with the accelerometer and display device, the controller configured to:
receive the respiratory rate data and the orientation data from the accelerometer;
determine based on the received respiratory rate data whether the respiratory rate of the patient is above a first predetermined threshold or below a second predetermined threshold;
determine based on the received orientation data whether the orientation of the patient above a predetermined threshold orientation;
trigger an alarm condition to the caregiver in response to at least one of:
a determination that the respiratory rate of the patient is above the first predetermined threshold;
a determination that the respiratory rate of the patient is below the second predetermined threshold; and
a determination that the orientation of the patient is above the predetermined threshold orientation,
the alarm condition comprising displaying, on the display, at least one pictograph indicating a course of action for the caregiver to take to correct the alarm condition;
a speaker configured to issue an audible alarm in response to a triggering of the alarm condition by the controller; and
a power source disposed within the housing, the power source providing energizing power to the accelerometer, controller, speaker and display device.

2. The physiology monitor as in claim 1, wherein the power source provides power to the physiology monitor for a minimum period of one year of continuous use.

3. The physiology monitor as in claim 1, wherein the patient is an infant.

4. The physiology monitor as in claim 1, wherein the patient is an adult.

5. The physiology monitor as in claim 1, wherein the at least one pictograph includes instructions for using the physiology monitor, and instructions for responding to alarm conditions issued by the controller.

6. The physiology monitor as in claim 1, wherein the housing is structured as a belt configured and dimensioned to encircle a chest region of the patient.

7. The physiology monitor as in claim 1, wherein the housing is structured as an adhesive fob configured to be removably adhered to a chest region of the patient.

8. The physiology monitor as in claim 1, further comprising a wireless communication circuit configured to receive telemetry data from at least one remote sensors, the at least one remote sensor located on a body regions of the patient.

9. The physiology monitor as in claim 8, wherein the at least one remote sensors is removably securable to the body region of the patient via at least one adhesive fob.

10. The physiology monitor as in claim 8, wherein the at least one remote sensor comprises at least one ring wearable on a finger or toe of the patient.

11. An infant monitor, comprising:
a housing comprising;
an accelerometer configured to:
monitor a respiratory rate of an infant and generate respiratory rate data based on the monitored respiratory rate of the infant; and
monitor an orientation of the infant and generate orientation data based on the monitored orientation of the infant;
an e-paper display disposed at a portion of the housing, the e-paper display device configured to display pictographic instructions to a caregiver;
a controller in communication with the accelerometer and display device, the controller configured to:
receive the respiratory rate data and the orientation data from the accelerometer;
determine based on the received respiratory rate data whether the respiratory rate of the patient is above a first predetermined threshold or below a second predetermined threshold;
determine based on the received orientation data whether the orientation of the patient above a predetermined threshold orientation;
trigger an alarm condition to the caregiver in response to at least one of:
a determination that the respiratory rate of the patient is above the first predetermined threshold;
a determination that the respiratory rate of the patient is below the second predetermined threshold; and
a determination that the orientation of the patient is above the predetermined threshold orientation.,
the alarm condition comprising displaying, on the display, at least one pictograph indicating a course of action for the caregiver to take to correct the alarm condition; and
and determining a risk to the infant based on the received signals;
a power source disposed within the housing, the power source providing energizing power to the accelerometer, controller and e-paper display.

12. The infant monitor as in claim 11, wherein the at least one pictograph includes instructions for using the infant monitor, and instructions for responding to alarm conditions issued by the controller.

13. The infant monitor as in claim 11, wherein the housing is structured as a belt configured and dimensioned to encircle a chest region of the infant.

14. The infant monitor as in claim 11, wherein the housing is structured as an adhesive fob configured to be removably adhered to a chest region of the infant.

15. The infant monitor as in claim 11, further comprising a wireless communication circuit configured to receive telemetry data from at least one remote sensor, the at least one remote sensor located on body region of the infant.

16. The infant monitor as in claim 15, wherein the at least one remote sensor is removably securable to the body region of the infant via at least one adhesive fobs.

17. The infant monitor as in claim 15, wherein the at least one remote sensor comprises at least one ring wearable on a finger or toe of the infant.

18. An infant monitor, comprising:
a belt dimensioned to encircle an infant's chest;
a detection means for monitoring a respiratory rate and an orientation of an infant, the detection means being disposed on the belt, the detection means configured to generate respiratory rate data based on the monitored respiratory rate of the infant and orientation data based on the monitored orientation of the infant;
a display means disposed at a portion of the belt, the display means configured to display pictographic instructions to a caregiver;
a control means in communication with the detection means and the display means, the controller configured for:
receiving the respiratory data and the orientation data from the detection means;
determining based on the received respiratory rate data whether the respiratory rate of the infant is above a first predetermined threshold or below a second predetermined threshold;
determining based on the received orientation data whether the orientation of the infant above a predetermined threshold orientation;
triggering an alarm condition to the caregiver in response to at least one of:
a determination that the respiratory rate of the infant is above the first predetermined threshold;
a determination that the respiratory rate of the infant is below the second predetermined threshold; and
a determination that the orientation of the infant is above the predetermined threshold orientation,
the alarm condition comprising displaying, on the display, at least one pictograph indicating a course of action for the caregiver to take to correct the alarm condition; and
a power source disposed within the belt, the power source providing energizing power to the detection means, control means and display means.

19. The physiology monitor as in claim 1, wherein the at least one pictograph displayed on the display comprises at least one of a pictograph indicating that the caregiver should pick up the patient and a pictograph indicating that the caregiver should turn over the patient.

20. The infant monitor as in claim 11, wherein the at least one pictograph displayed on the display comprises at least one of a pictograph indicating that the caregiver should pick up the infant and a pictograph indicating that the caregiver should turn over the infant.
